(19) United States
(12) Patent Application Publication
(10) Pub. No.: US 2009/0131906 A1
(43) Pub. Date: May 21, 2009

(54) METHOD, SYSTEM, APPARATUS, AND KIT FOR REMOTE THERAPEUTIC DELIVERY

(76) Inventors: Toby FREYMAN, Waltham, MA (US); Maria Palasis, Wellesley, MA (US); Timothy J. Mickley, Elk River, MN (US)

Correspondence Address: KENYON & KENYON LLP, 1500 K STREET N.W., SUITE 700, WASHINGTON, DC, 20005, US

(21) Appl. No.: 12/358,337

(22) Filed: Jan. 23, 2009

Related U.S. Application Data
(62) Division of application No. 11/155,603, filed on Jun. 20, 2005.

(51) Int. Cl. A61M 25/10

(52) U.S. Cl. 604/505; 604/509; 604/503

(57) ABSTRACT

A therapeutic delivery catheter system method or kit for delivery of therapeutic to a target location is provided. In various embodiments of the present invention the invention may include a catheter with a therapeutic delivery lumen and a therapeutic delivery orifice. The lumen and the orifice may be in fluid communication with each other and may be configured such that therapeutic delivered therethrough may be done at pressures mimicking pressures existing or otherwise normal at the target locations receiving the therapeutic. In some embodiments the catheter may be part of a kit that may include instructions regarding the proper manner of operation of the catheter. These instructions may include suggested target pressures for therapeutic delivery as well as delivery times, and suggested lengths of time for the device to reside at the target area after delivery to allow for proper uptake of the therapeutic.

### FIELD OF THE INVENTION

The present invention is directed to therapeutic delivery. More specifically, the present invention is directed to systems, methods, apparatus, and kits that may be used or employed to deliver therapeutic through a lumen to a target site remote from a medical practitioner performing the procedure.

### BACKGROUND OF THE INVENTION

Contemporary medical procedures often involve the delivery of therapeutic to target sites located within the body of a patient. These target sites may be accessible through the various lumens of the body as well as through techniques that do not employ passage through a lumen of the body. Typical target sites within the body may include the organs and vessels of the body as well as any other site or area that may benefit from being interfaced with a therapeutic. In some instances, the target site may be located outside of the patient, such as when a donor organ is maintained prior to implantation.

When therapeutic is delivered through a lumen of a medical device, the therapeutic is often forced through the lumen of the device prior to its ejection and delivery to a target site. In some instances, a practitioner will force the therapeutic through the medical device by depressing a plunger coupled to a proximal portion of the medical device. As the plunger is depressed, the therapeutic will be urged, under pressure, through the lumen until it's discharged from the lumen.

### BRIEF DESCRIPTION OF THE DRAWINGS

FIG. 1 is a cross-section of a medical device having reduced internal friction in accord with an embodiment of the present invention.

FIG. 2 is a table showing internal vessel pressures that may be developed as therapeutic is delivered in accord with an embodiment of the present invention.

FIG. 3 is a catheter having a proximal coupling hub in accord with an embodiment of the present invention.

FIG. 4 is the distal end of a catheter located within a coronary vessel in accord with an embodiment of the present invention.

FIG. 5 is the distal end of a catheter that employs an expandable balloon in accord with an embodiment of the present invention.

FIG. 6 is a coupling hub of a medical device that may be used in accord with an embodiment of the present invention.

FIG. 7 is a cross-sectional view of a catheter that may be employed in accord with an embodiment of the present invention.

FIG. 8 is across-section view of a catheter that may be employed in accord with an embodiment of the present invention.

FIG. 9 is the distal end of a catheter that employs an expandable balloon in accord with an embodiment of the present invention.

FIG. 10 is the distal end of the catheter from FIG. 9 with the balloon in a first position.

FIG. 11 is the distal end of the catheter from FIG. 9 with the balloon in a second position.

FIG. 12 is a cross-sectional view of a medical device that may be employed in accord with an embodiment of the present invention.

FIG. 13 is a side view of a balloon catheter in accord with an embodiment of the present invention.

FIG. 14 is a side view of a distal end of a balloon catheter that may be employed in accord with the present invention.

FIG. 15 is a table reflecting the number of cells that remain in an infarct following a fourteen day period using various delivery techniques or devices in accord with an embodiment of the present invention.

### DETAILED DESCRIPTION

A therapeutic delivery catheter system method or kit for delivery of therapeutic to a target location is provided. In various embodiments of the present invention the invention may include a catheter with a therapeutic delivery lumen and a therapeutic delivery orifice. The lumen and the orifice may be in fluid communication with each other and may be configured such that therapeutic delivered therethrough may be done at pressures mimicking pressures existant or otherwise normal at the target locations receiving the therapeutic. In some embodiments the catheter may be part of a kit that may include instructions regarding the proper manner of operation of the catheter. These instructions may include suggested target pressures for therapeutic delivery as well as delivery times, and suggested lengths of time for the device to reside at the target area after delivery to allow for proper uptake of the therapeutic.

A typical target site may be within the body of a patient and may include the coronary vasculature of a patient as well as various organs within the body of a patient. A target site may also be other systems, organs, and tissues, both within and outside of the body.

In one embodiment of the present invention, a lumen of a delivery device, such as a catheter, may be sized to reduce the amount of internal fluid resistance opposing a therapeutic as it is delivered through the lumen, to a target site. In so doing, the therapeutic may be delivered by the device at pressures more effective than those in the past. The therapeutic may be delivered more quickly and with more tactile feedback as well. The pressures developed may be sufficient to influence the movement of cells in the myocardium or other target area; these pressures may be in the range of a patient's systolic pressure to a patient's diastolic pressure (80-120 mm Hg, more or less).

As noted, the therapeutic may be delivered in a fashion that provides tactile feedback to a practitioner as the therapeutic is delivered. This tactile feedback may include forces generated by the target area opposing the delivery of therapeutic as the target area receives therapeutic during the procedure. This tactile feedback may also be caused by other sources at the target site as well. Through embodiments of the present invention, the amount of engrafted cells delivered to a target site as therapeutic may be increased. Other therapeutics may also be more efficiently delivered through use of the present invention.

The present invention may be embodied in various systems, methods, apparatus, and kits including those described herein. Moreover, the present invention may not only be embodied by the described embodiments but it may also be embodied by various combinations of these embodiments, which may or may not substitute one or more features or processes in one embodiment for features or processes of another embodiment. Still further, techniques involving the present invention may include those described herein, others performed in differing order, and combinations of the described techniques as well. Moreover, these and various other steps may be described as instructions for kits employing the present invention.

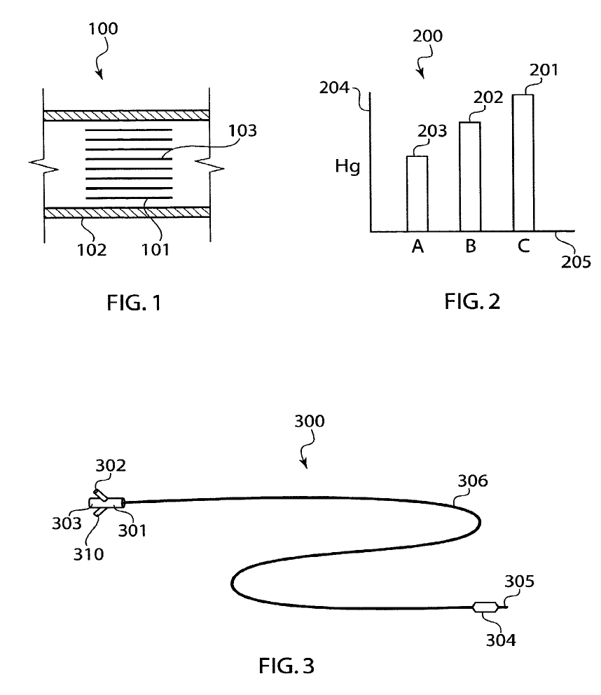

FIG. 1 is a cross-section of a lumen 102 that may be within a medical device 100 in accord with an embodiment of the present invention. Shown within the lumen 102 are flow lines 101 and 103 indicating the direction of flow of a fluid flowing within the lumen 102. The length of these flow lines 101 and 103 reflect the relative rate of flow of fluid traveling through the lumen 102. Accordingly, as can be seen, the flow of a fluid near the center of lumen 102 is more rapid than the flow of therapeutic near the inner surfaces of the lumen 102. By reducing the opposing friction or resistance associated with the movement of the fluid within the lumen 102: (a) the overall speed of the fluid moving through may be increased; (b) the difference in fluid speed across the cross-section of the lumen may be reduced; (c) the amount of force needed to urge the fluid through the lumen 102 may be reduced; (d) the amount of tactile response to a practitioner at the proximal end of the device may be improved; and (e) the magnitude of the delivery pressures generated at the distal end of device 100 may be increased.

FIG. 2 is table 200 that reflects delivery pressures that may be developed at a distal end of a medical device in accord with an embodiment of the present invention. Pressure in mm Hg is generally reflected along the y-axis 204 while three distinct pressures are located on the x-axis 205. Bars 201 and 203 reflect the systolic and diastolic pressures that may exist in the cardiovascular system of a patient, while bar 202 reflects the pressure that may be developed in a fluid delivered to a target site in accord with an embodiment of the present invention. As can be seen, the pressure 202 generated is between the systolic and diastolic pressures of the patient in this example. In other embodiments, however, the delivery pressure 202 may be above pressure 201 or below pressure 203. Nevertheless, in a preferred embodiment, the delivery pressure 202 will not exceed the larger pressure 201 of the patient. By delivering therapeutic at the illustrated delivery pressure 202 the therapeutic may be readily taken up by the target site, which in this example are vessels within the cardiovascular system.

Optimum delivery pressures may also be determined by considering a patient's systole and diastole pressure. These measurements may disclose the max pressure that the patient's vessels may withstand. An EKG may also be used to determine the timing of when to deliver the therapeutic and the duration that the therapeutic may be delivered. For example, during times of rest, a therapeutic may be delivered and during times of compression, therapeutic delivery may cease. In a preferred embodiment, flow rates and pressures of therapeutic will mimic those naturally present in the body. For instance, they may plateau at 120 mm Hg at 56 cc/min. In so doing, an adequate amount of cells or other therapeutic may be delivered and driven into the tissue at the target site.

FIG. 3 is a medical device in accord with an embodiment of the present invention. The catheter 300 of FIG. 3 includes: a coupler 301, with hubs 302 and 303 and one or more lumens within member 306; an expandable balloon 304; and a distal delivery end 305. In this embodiment the hubs 302 and 303 may be coupled to a source of therapeutic and a push fluid hub 310 may also be coupled to another type of balloon inflation media. In use, the therapeutic may be urged through the member by the push fluid until it emerges from the distal delivery end 305 of the device. The lumen within member 306 may be sized such that little resistance is generated against the movement of the therapeutic and fluid out the distal delivery end 304 of the catheter 300. This lumen may be sized greater than 0.4 mm in diameter so that the flow rates, pressures, and volumes of the delivered therapeutic may be physiologically relevant, which may include reaching pressures of 100 mm Hg at 28 cc/min. These delivery pressures may be measured and monitored by sensors located along the medical device, including sensors located at the distal end of the catheter 300. This medical device, like the other embodiments, may be part of a kit that may be distributed to medical institutions and medical practitioners, the kit including instructions that describe the use of the device, including some or all of the steps described herein.

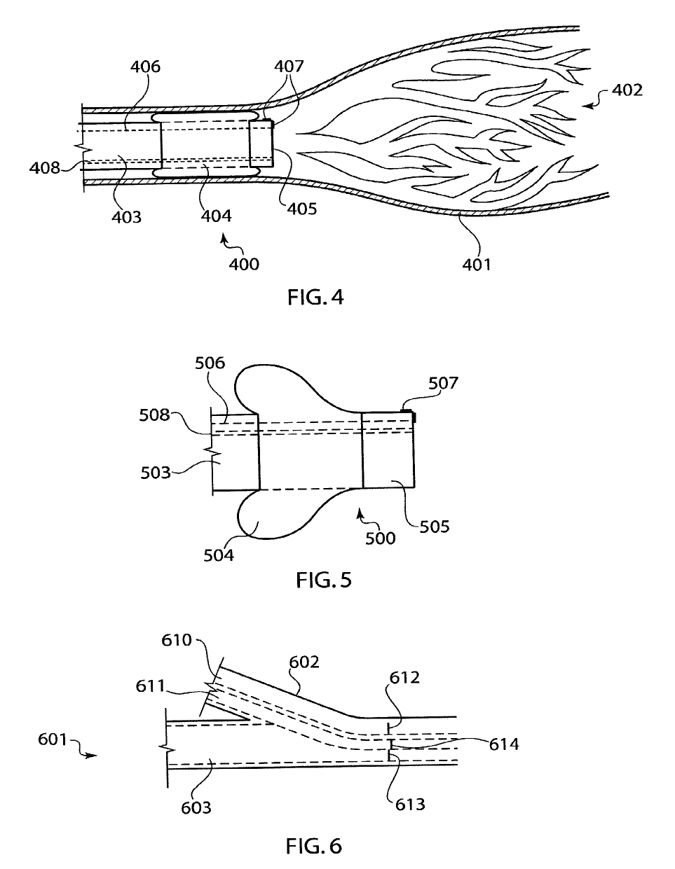

FIG. 4 shows a delivery device 400 after it has been positioned within a vessel 402 of a patient, as may occur in an embodiment of the present invention. The device 400, in FIG. 4, is a catheter with sensors 407, sensor line 406, lumen 403, delivery end 405, guide-wire lumen 408, and balloon 404. The device 400 has been positioned near the target vessel 402, as may be done in accord with an embodiment of the present invention. The device may have been positioned by sliding it over a guide-wire located within guide-wire lumen 408. It may have been positioned with other methods as well. Once properly positioned, the balloon 404 may be inflated and therapeutic, followed by a pushing fluid, may be urged through lumen 403. Upon exiting the distal end 405, the sensors 407 may monitor various parameters including the pressure, flow rate, and volume of therapeutic and pushing fluid entering the vessel 402. Due to the pressures, flow rates, and volumes generated by the present invention, therapeutic exiting the device 400 may be easily and readily delivered to the target site 402. Moreover, when the flow rates are increased, the tactile response available to a practitioner may be increased. These flow rates and reduced pressures may be increased by providing a lumen with an internal diameter of 0.4 mm in diameter or more. The flow rates may be measured by using doppler echo techniques.

In use, a practitioner may deliver therapeutic through the device and then wait a predetermined amount of time, such as two minutes before withdrawing the device. In embodiments that include an inflation balloon, the balloon may first be inflated before the therapeutic is delivered under pressure. The balloon will occlude the vessel and stop therapeutic from being delivered proximal of the balloon, in order to elevate the pressure in the vessel to be closer to the delivery pressures generated in the lumen of the delivery device. During this time, as well as during other periods, the pressure or other parameters of the target area may be measured or monitored. In some embodiments, the delivery orifice of the device may be positioned well upstream of the target site such that the therapeutic may be delivered to the target site through lumens of the body at pressures supplemented by the delivery device. Furthermore, rather than using a single lumen to carry both the therapeutic and the flushing fluid, multiple lumens may be used, with each lumen carrying one or more of these materials. In other embodiments, a booster, such as an oxygenated medium may also be used to affect the delivery and uptake of therapeutic at the delivery site. This oxygenated medium (or uptake booster) may include a cell suspension as well as anti-oxidents, nutrients, vasodilators, vasoconstrictors, contrast mediums, and saline as well as various combinations of these and other materials.

FIG. 5 is a side view of a distal end of a delivery catheter 500 in accord with an embodiment of the present invention. In this figure, lumen 503, sensors 507, sensor line 506, guide-wire lumen 508, distal end 505, and balloon 504 are all accordingly labeled. The balloon 504 in this embodiment is shown in a partially expanded state. As can be seen, the balloon 504 is somewhat conically shaped having a smaller leading area and a larger trailing area. Thus, as the device 500 is urged into a target area the balloon 504 may form a tighter seal the further the device 500 is urged into the target area. The balloon may have other shapes as well. Moreover, this balloon, as well as others in accord with the present invention, may also be made of materials permeable to oxygen. Using these materials may be advantageous when the balloon is inflated with an oxygenated material, as this may reduce ischemia of the endothelium.

FIG. 6 shows a coupler 601 that may be employed in accord with an embodiment of the present invention. This coupler 601 may be used in the device of FIG. 3, as well as in other devices. This coupler 601 includes a first lumen 610, a second lumen 611, and a third lumen 603. Each of these lumens contains a one-way valve 612, 613, and 614 to prevent fluid from returning upstream through the coupler. These one-way valves may be flaps sized to maintain pressures downstream of the flap. The valves may be constructed in different ways and may also include other functions, such as metering and timed release features, in addition to or in place of a one-way valve feature.

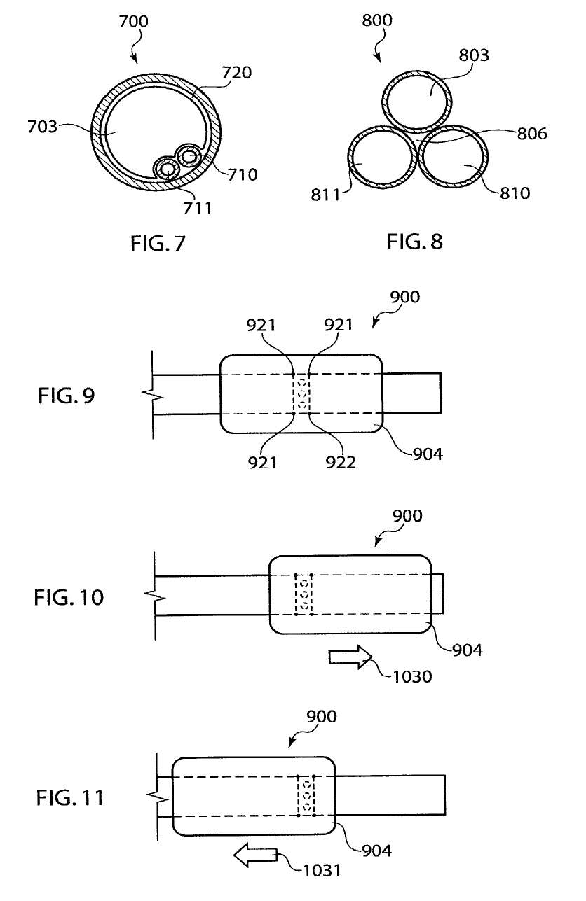

FIG. 7 is a cross-section of a catheter 700 in accord with an embodiment of the present invention. This catheter 700 may include a lining or treatment 720, a central lumen 703, and secondary lumens 710 and 711. The lining 720 may be chosen to be compatible with a therapeutic or other material that may travel through the catheter 700. The secondary lumens may be used to deliver a guide-wire and to inflate a balloon coupled to the catheter.

FIG. 8 is a cross-section of a medical device 800 in accord with another embodiment of the present invention. The device 800 in FIG. 8 includes three lumens 803, 811, and 810 stellately positioned next to one another such that a channel 806 is formed. This channel may be used to surround a guide-wire that may be used to position the medical device 800 during a procedure.

FIGS. 9-11 show a medical device in accord with an alternative embodiment of the present invention. Labeled in these figures are catheter 900, inflation ports 922, balloon 904, securement points 921, and movement arrows 1030 and 1131. In this embodiment, the balloon 904 may be secured to the catheter 900 at points 921. In so doing, the catheter may slide relative to the balloon should the need arise during a procedure. In other words, once the balloon is expanded at a target site, should an unwanted longitudinal force be placed on the catheter, rather than moving the balloon and the catheter, only the catheter may initially move, sliding within the balloon. Arrows 1030 and 1131 show the movement of the balloon relative to the catheter. The balloon may be shaped in accord with the balloons shown above.

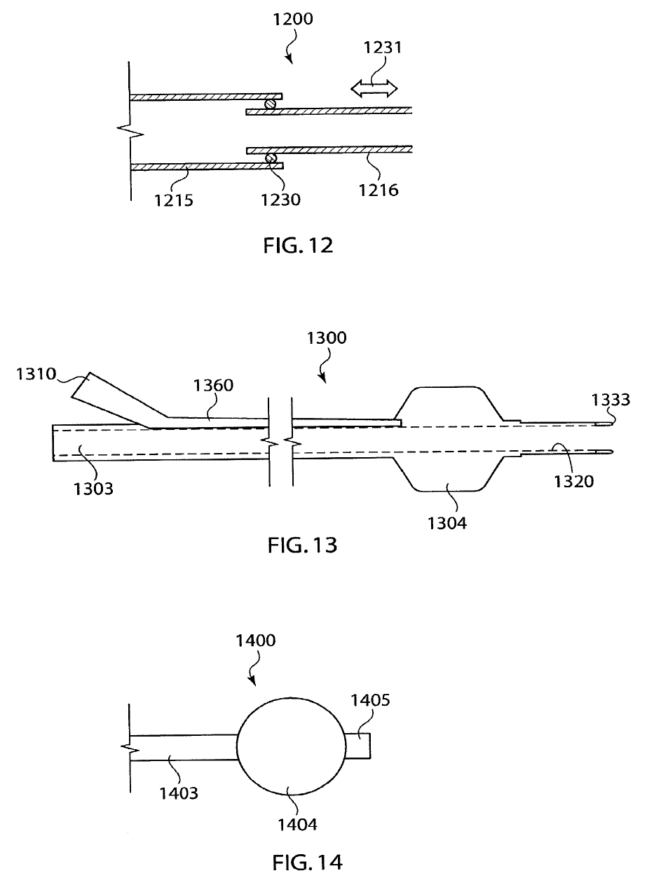

FIG. 12 shows a linking detail that may be used in the catheter of FIG. 3 as well as in the other devices of the present invention. The coupling or linking technique may include a joint 1200 including a first section 1215 and a second section 1216. Sealing the first and second sections may be ring or gasket 1230. By using this joist in a medical device, unwanted longitudinal forces that can damage a vessel wall may be buffered and not readily transmitted up or down the device.

FIG. 13 is a balloon catheter 1300 in accord with an embodiment of the present invention. This balloon catheter may include a delivery lumen 1303, an inflation hub 1310, an inflation lumen 1360, a balloon 1304, a therapeutic liner 1320, and an atraumatic tip 1333.

FIG. 14 is a distal end of another embodiment of the present invention. The medical device 1400 of this embodiment includes a delivery lumen 1403, a spherical balloon 1404, and a distal delivery end 1405.

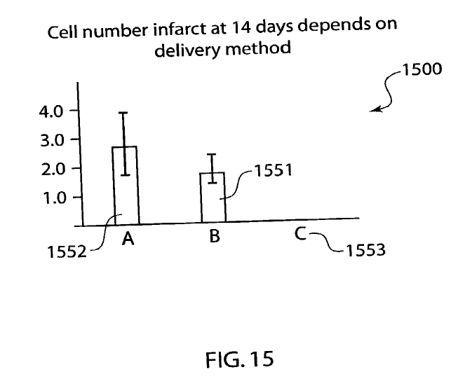

FIG. 15 is a table showing cell number infarct at 14 days depending on delivery method. In the first column an intracoronary method is shown. The second column shows an endocardial method and the third column shows an intravenous method. As can be seen, the intravenous method was ineffective and the intracoronary method was the most effective. Accordingly, it has been found that intracoronary infusion of cells, in a porcine model of AMI, results in more engrafted cells in the myocardium as compared to the same dose of cells directly injected into the tissue.

Several therapeutics or drugs that may be delivered in accord with the present invention. The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents;

agents which interfere with endogeneus vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation. Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin⁻) cells including Lin⁻ CD34⁻, Lin⁻CD34⁺, Lin⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts +5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Any of the above mentioned therapeutic agents may be incorporated into a polymeric coating. The polymers of the polymeric coatings may be biodegradable or non-biodegradable. Non-limiting examples of suitable non-biodegradable polymers include polystrene; polyisobutylene copolymers and styrene-isobutylene-styrene block copolymers such as styrene-isobutylene-styrene tert-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Such coatings used with the present invention may be formed by various methods. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent/therapeutic agent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

1-22. (canceled)

23. A method for delivering a therapeutic to a target location in a patient comprising:
providing a balloon catheter comprising a therapeutic delivery lumen, a balloon, and a distal end,
positioning the balloon catheter in a body lumen with the distal end at the target location,
inflating the balloon to occlude the body lumen,
measuring a pressure at the target location, and
delivering a therapeutic at the measured pressure.

24. The method of claim 23, further comprising delivering a pushing fluid at the measured pressure.

25. The method of claim 23, further comprising monitoring the delivery pressure of the therapeutic exiting the catheter.

26. The method of claim 23, further comprising delivering an oxygenated medium or uptake booster.

27. The method of claim 23, further comprising performing an EKG on the patient.

28. The method of claim 27, further comprising delivering the therapeutic during times of rest of the heart and ceasing delivery of the therapeutic during times of compression of the heart based on the EKG results.

29. The method of claim 23, further comprising setting a maximum pressure for delivering the therapeutic.

30. The method of claim 23, further comprising setting a minimum pressure for delivering the therapeutic.

31. The method of claim 23, wherein the therapeutic comprises cells.

32. The method of claim 23, wherein the target location is a myocardium.

33. The method of claim 23, further comprising waiting a predetermined amount of time before withdrawing the balloon catheter from the lumen.

34. The method of claim 23, further comprising measuring the pressure with a pressure sensor.

35. The method of claim 23, further comprising measuring the patient's systole and diastole pressure prior to delivering the therapeutic.

36. The method of claim 23, wherein the therapeutic delivery lumen has an internal diameter along more than half the length of the lumen of 0.4 mm.

37. The method of claim 23, wherein the balloon has an expanded shape with a low profile side and a high profile side, the low profile side being closer to the distal end of the catheter than the high profile side.

38. The method of claim 34, wherein the pressure sensor is a Doppler echo sensor.

39. The method of claim 23, further comprising delivering a pushing fluid after delivering the therapeutic.

40. The method of claim 39, wherein the pushing fluid is saline.

41. The method of claim 23, wherein the therapeutic delivery lumen comprises a first section and a second section, the first section being slidable within the second section.

42. The method of claim 41, wherein the catheter comprises a gasket positioned between the first section and the second section to provide a seal.
